(19) United States
(12) Patent Application Publication
(10) Pub. No.: US 2023/0338090 A1
(43) Pub. Date: Oct. 26, 2023

(54) Fiber Optic Guidewire Sensing Systems and Methods

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 17/725,394

(22) Filed: Apr. 20, 2022

(51) Int. Cl. A61B 5/283; A61B 5/339; A61B 5/06; A61B 5/11; A61B 34/20; A61B 5/00

(52) U.S. Cl. CPC A61B 34/20; A61B 5/068; A61B 5/1126; A61B 5/283; A61B 5/339; A61B 5/6851; A61B 2034/2061

(57) ABSTRACT

Disclosed herein are medical systems and devices that include an elongate probe configured for insertion into a patient and/or a catheter. The elongate probe includes an optical fiber and a number of electrical conductors extending along the elongate probe. The probe further includes a tip electrode and a number of band electrodes for obtaining an ECG signal and a bioimpedance, respectively. The optical fiber is configured for shape sensing and to detect strain and fluctuations of the probe. A system includes the probe, and a logic of the system is configured to determine a location of the probe along a vasculature and further determine a location of a catheter with respect to the probe via optical signals and/or electrical signals.

### BACKGROUND

Elongate medical devices configured for insertion with a patient may be utilized to perform a myriad of treatments and diagnoses. An elongate medical device may be a catheter that is advanced along a vasculature of the patient to deliver medication to the patient at a desired location of the vasculature, such as the superior vena cava, for example. As such, proper placement of the catheter along the vasculature may be important and improper placement may define a risk to the patient. Some medical devices include electrical conducting members extending along the length of the medical device. One such system is disclosed in U.S. Pat. No. 8,801,693, titled “Bioimpedance-Assisted Placement of a Medical Device” filed Oct. 27, 2011, which is incorporated herein by reference in its entirety. Some elongate devices may include fiber optic capability.

Disclosed herein are medical devices and systems that include fiber optic capability and electrical capability that address the forgoing.

### SUMMARY

Briefly summarized, disclosed herein is a medical device. According to some embodiments, the medical device includes an elongate probe configured for insertion into a patient body, where the elongate probe defines a proximal end and a curved distal tip at a distal end. The device further includes an optical fiber extending along the elongate probe from the proximal end to the distal end, where the optical fiber includes one or more core fibers extending along a longitudinal length of the optical fiber. Each of the one or more core fibers includes a plurality of sensors distributed along the longitudinal length and each sensor of the plurality of sensors is configured to (i) reflect a light signal of a different spectral width based on received incident light at proximal end, and (ii) change a characteristic of the reflected light signal based on a condition experienced by the optical fiber along the curved distal tip.

In some embodiments, the elongate probe is configured for advancement along a vasculature of the patient body such that the elongate probe experiences fluctuations due to fluctuating movement of patient body tissue, and the fluctuations define the condition experienced by the optical fiber along the curved distal tip.

In some embodiments, the elongate probe is configured for insertion within a lumen of a vascular catheter, and the curved distal tip includes a flexibility in bending such that, upon disposition of the curved distal tip within the lumen, a radius of curvature of the curved distal tip is increased. The increase in the radius of curvature defines a bending strain along the curved distal tip, and the bending strain defines the condition experienced by the optical fiber along the curved distal tip.

In some embodiments, the elongate probe includes a guidewire.

In some embodiments, the elongate probe includes a number of electrical conductors extending along the elongate probe from the proximal end to the distal end.

In some embodiments, the elongate probe includes a tip electrode at the distal end, where the tip electrode is coupled with at least one of the number of electrical conductors, and where the tip electrode is configured to obtain an ECG signal from the patient body.

In some embodiments, the elongate probe includes a number of band electrodes disposed along the elongate probe, where each band electrode is coupled with at least one of the number of electrical conductors, and where the band electrodes are configured to obtain an electrical impedance between two or more band electrodes.

Also disclosed herein is a medical system that includes a medical device. The medical device includes an elongate probe configured for insertion into a patient body, where the elongate probe defines a proximal end and a curved distal tip at a distal end. An optical fiber extends along the elongate probe from the proximal end to the distal end, where the optical fiber includes one or more core fibers extending along a longitudinal length of the optical fiber. Each of the one or more core fibers includes a plurality of sensors distributed along the longitudinal length and each sensor of the plurality of sensors is configured to (i) reflect a light signal of a different spectral width based on received incident light at proximal end, and (ii) change a characteristic of the reflected light signal based on condition experienced by the optical fiber. The system further includes a console operatively coupled with the medical device at the proximal end, where the console includes one or more processors, and a non-transitory computer-readable medium having stored thereon logic that, when executed by the one or more processors, causes operations of the system. The operations include determining a physical state of the curved distal tip, where determining the physical state includes: (i) providing an incident light signal to the optical fiber; (ii) receiving reflected light signals of different spectral widths of the incident light by one or more of the plurality of sensors along the curved distal tip; and (iii) processing the reflected light signals associated with the one or more core fibers to determine the physical state of the curved distal tip.

In some embodiments of the system, the physical state includes fluctuations along the curved distal tip, the fluctuations caused by fluctuating tissue movement within the patient body and in some embodiments, the fluctuating tissue movement is caused by a heartbeat.

In some embodiments of the system, the physical state includes a bending strain along the curved distal tip and in some embodiments, the bending strain along the curved distal tip is caused by disposition of the curved distal tip within a lumen of a vascular catheter.

In some embodiments of the system, the elongate probe includes a number of electrical conductors extending along the elongate probe from the proximal end to the distal end, and the operations of the system include receiving an electrical signal from one or more of the electrical conductors.

In some embodiments of the system, the elongate probe includes a tip electrode at the distal end, where the tip electrode is coupled with at least one of the number of electrical conductors, and where the electrical signal includes an ECG signal.

In some embodiments of the system the elongate probe includes a number of band electrodes disposed along the elongate probe, where each band electrode is coupled with at least one of the number of electrical conductors, and where the electrical signal includes an impedance between two or more of the number of band electrodes.

Also disclosed herein is a method of placing a catheter within a vasculature of a patient body. According to some embodiments, the method includes providing a guidewire, where the guidewire includes an optical fiber extending along the guidewire, and where the optical fiber is operatively coupled with a console. the guidewire further includes a number of electrical conductors extending along the guidewire, where the electrical conductors are operatively coupled with the console. The method further includes (i) advancing the guidewire along a vascular pathway of the patient body and (ii) determining a position of the guidewire within the vascular pathway based on one or more of a first optical signal received by the console from the guidewire or a first electrical signal received by the console from the guidewire. The method further includes (i) advancing the catheter along the guidewire, where the guidewire is disposed within a lumen of the catheter and (ii) determining a location of the catheter with respect to the guidewire based on one or more of a second optical signal or a second electrical signal.

In some embodiments of the method, the optical fiber includes one or more core fibers extending along a longitudinal length of the optical fiber, where each of the one or more core fibers includes a plurality of sensors distributed along the longitudinal length, and where each sensor of the plurality of sensors is configured to (i) reflect a light signal of a different spectral width based on received incident light at proximal end, and (ii) change a characteristic of the reflected light signal based on condition experienced by the optical fiber. In such embodiments, the first optical signal includes reflected light signals of different spectral widths from one or more of the plurality of sensors based on received incident light at the proximal end, where the different spectral widths are defined by a fluctuation of the optical fiber, and where the fluctuation is caused by fluctuating tissue movement adjacent the vascular pathway.

In some embodiments of the method, the guidewire includes a curved distal tip and the second optical signal includes reflected light signals of different spectral widths from one or more of the plurality of sensors disposed along the curved distal tip based on received incident light at the proximal end, where the different spectral widths are defined by a bending strain along the curved distal tip, and where the bending strain is caused by advancing the catheter along the curved distal tip.

In some embodiments of the method, the guidewire includes a tip electrode at the distal end, where the tip electrode is coupled with at least one of the number of electrical conductors. In such embodiments, the first electrical signal includes an ECG signal obtained by the tip electrode.

In some embodiments of the method, the guidewire includes a plurality of band electrodes disposed along the guidewire, where each band electrode is coupled with at least one of the number of electrical conductors. In such embodiments, the first electrical signal is defined by an electrical impedance between two or more band electrodes, where the electrical impedance is defined by a change in an annular fluid pathway extending along the two or more band electrodes, where the change in the annual fluid pathway is caused by advancing the guidewire between two portions of the vascular pathway, and where the two portions have different cross-sectional areas.

In some embodiments of the method, the guidewire includes a plurality of band electrodes disposed along the guidewire, where each band electrode is coupled with at least one of the number of electrical conductors. In such embodiments, the second electrical signal is defined by an electrical impedance between two or more band electrodes, where the electrical impedance is defined by a change in an annular fluid pathway extending along the two or more band electrodes, and where the change in the annual fluid pathway is caused by advancing the catheter over the two or more band electrodes.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustrative embodiment of a medical device placement system including a medical device with fiber optic and electrical capabilities, in accordance with some embodiments;

FIG. 2 is an exemplary embodiment of a structure of the elongate probe of FIG. 1, in accordance with some embodiments;

FIG. 3A illustrates an embodiment of the elongate probe of FIG. 1, in accordance with some embodiments;

FIG. 3B is a cross sectional view of the elongate probe of FIG. 3A, in accordance with some embodiments;

FIGS. 4A-4B are flowcharts of methods of operations conducted by the medical device system of FIG. 1 to achieve optical three-dimensional shape sensing, in accordance with some embodiments;

FIG. 5 illustrates an exemplary embodiment of the medical instrument placement system 100 of FIG. 1 during operation and insertion of the elongate probe within a patient, in accordance with some embodiments;

FIG. 6A illustrates an impedance between two band electrodes of distal portion of the elongate probe of FIG. 5 disposed within a first blood vessel of the patient, in accordance with some embodiments;

FIG. 6B illustrates an impedance between the two band electrodes of distal portion a distal portion disposed within a second blood vessel of the patient, in accordance with some embodiments;

FIG. 6C illustrates an impedance between the two band electrodes of distal portion a distal portion disposed within a lumen of the catheter of FIG. 5, in accordance with some embodiments;

FIG. 7A illustrates a curved distal tip of the elongate probe of FIG. 5 disposed outside of the lumen of the catheter of FIG. 5, in accordance with some embodiments; and

FIG. 7B illustrates the curved distal tip of the elongate probe disposed within the lumen of the catheter, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit (ASIC), etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

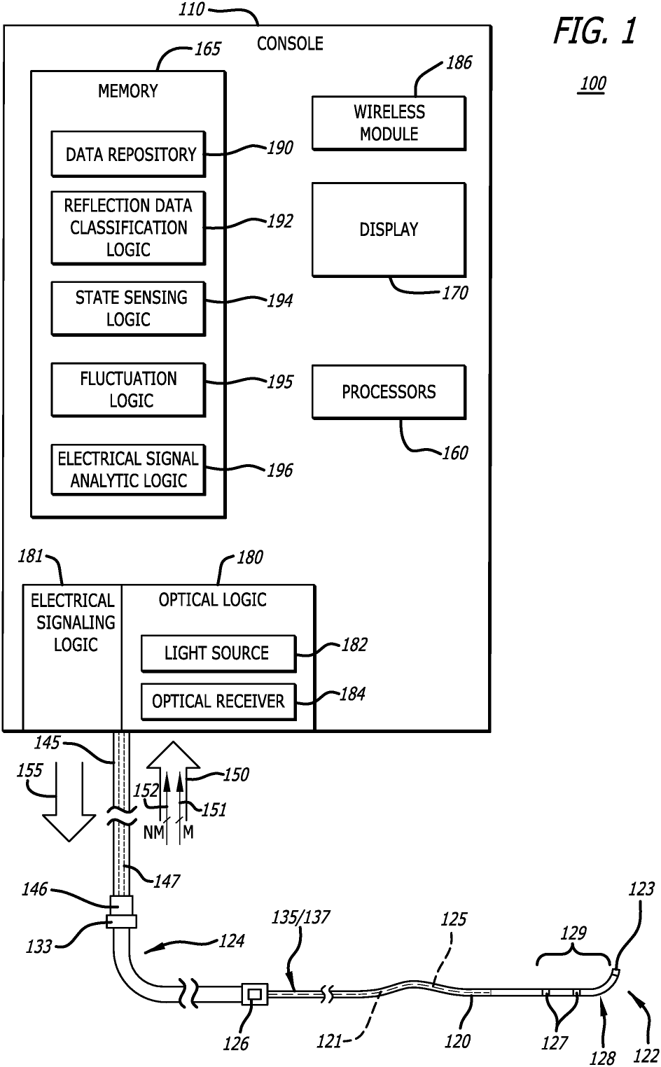

FIG. 1 illustrates an embodiment of a medical instrument placement system including a medical instrument. As shown, the medical instrument placement system (system) 100 generally includes a console 110 and an elongate probe 120 communicatively coupled with the console 110. The elongate probe 120 defines a distal end 122 and includes a console connector 133 at a proximal end 124. The elongate probe 120 includes an optical fiber 135 including multiple core fibers extending along a length of the elongate probe 120 as further described below. The console connector 133 enables the elongate probe 120 to be operably connected to the console 110 via an interconnect 145 including one or more optical fibers 147 (hereinafter, “optical fiber(s)”). The elongate probe 120 further includes a number of electrical conductors 125 (e.g., wires) that extend along the elongate probe 120. The electrical conductors 125 may define an electrical coupling of a tip electrode 123 at the distal end 122 to a single optical/electric connector 146 (or dual connectors) at the proximal end 124. Herein, the connector 146 is configured to engage (mate) with the console connector 133 to allow for the propagation of light between the console 110 and the elongate probe 120 as well as the propagation of electrical signals from the elongate probe 120 to the console 110. The tip electrode 123 may be configured to obtain an electrical signal from the patient (e.g., an ECG signal). The elongate probe 120 may include a number of band electrodes 127 disposed along an outer surface of the elongate probe 120 and the electrical conductors 125 may define an electrical coupling of the band electrodes 127 to the optical/electric connector 146. For illustrative purposes, a distal portion 129 of the elongate probe 120 is defined that includes the curved distal tip 128, the tip electrode 123, and the band electrodes 127.

The elongate probe 120 includes a curved distal tip 128. The curved distal tip 128 may define a curved shape in a free state. The curved distal tip 128 may include a bending flexibility to allow the curved shape to straighten, i.e., become less curved during use, such as when the curved distal tip 128 is disposed within a lumen of a catheter, for example.

The elongate probe 120 may be configured to perform any of a variety of medical procedures. As such, the elongate probe 120 may be a component of or employed with a variety of medical instruments/devices. In some implementations, the elongate probe 120 may take the form of a guidewire or a stylet, for example. The elongate probe 120 may be formed of a metal, a plastic or a combination thereof. The elongate probe 120 includes a lumen 121 extending therealong having an optical fiber 135 disposed therein.

In some implementations, the elongate probe 120 may be employed with a vascular catheter. Other exemplary implementations include drainage catheters, surgery devices, stent insertion and/or removal devices, biopsy devices, endoscopes, and kidney stone removal devices. In short, the elongate probe 120 may be employed with, or the elongate probe 120 may be a component of, any medical device that is inserted into a patient.

According to one embodiment, the console 110 includes one or more processors 160, a memory 165, a display 170, and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The one or more processors 160, with access to the memory 165 (e.g., non-volatile memory or non-transitory, computer-readable medium), are included to control functionality of the console 110 during operation. As shown, the display 170 may be a liquid crystal diode (LCD) display integrated into the console 110 and employed as a user interface to display information to the clinician, especially during an instrument placement procedure. In another embodiment, the display 170 may be separate from the console 110. Although not shown, a user interface is configured to provide user control of the console 110.

According to the illustrated embodiment, the content depicted by the display 170 may change according to which mode the elongate probe 120 is configured to operate: optical, TLS, ECG, or another modality. In TLS mode, the content rendered by the display 170 may constitute a two-dimensional or three-dimensional representation of the physical state (e.g., length, shape, form, and/or orientation) of the elongate probe 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below.

According to one embodiment of the disclosure, an activation control 126, included on the elongate probe 120, may be used to set the elongate probe 120 into a desired operating mode and selectively alter operability of the display 170 by the clinician to assist in medical device placement. For example, based on the modality of the elongate probe 120, the display 170 of the console 110 can be employed for optical modality-based guidance during probe advancement through the vasculature or TLS modality to determine the physical state (e.g., length, form, shape, orientation, etc.) of the elongate probe 120. In one embodiment, information from multiple modes, such as optical, TLS or ECG for example, may be displayed concurrently (e.g., at least partially overlapping in time).

Referring still to FIG. 1, the optical logic 180 is configured to support operability of the elongate probe 120 and enable the return of information to the console 110, which may be used to determine the physical state associated with the elongate probe 120. Electrical signals, such as ECG signaling, may be processed via an electrical signaling logic 181 that supports receipt and processing of the received electrical signals from the elongate probe 120, (e.g., ports, analog-to-digital conversion logic, etc.). Electrical signals, such as a pacemaker signal, for example, may also be defined and provided by the electrical signaling logic 181. The physical state of the elongate probe 120 may be based on changes in characteristics of the reflected light signals 150 received at the console 110 from the elongate probe 120. The characteristics may include shifts in wavelength caused by strain along certain regions of the core fibers integrated within the optical fiber 135 positioned within or operating as the elongate probe 120, as shown below. As discussed herein, the optical fiber 135 may be comprised of core fibers 137₁-137_{M} (M=1 for a single core, and M>2 for a multi-core), where the core fibers 137₁-137_{M} may collectively be referred to as core fiber(s) 137. Unless otherwise specified or the instant embodiment requires an alternative interpretation, embodiments discussed herein will refer to an optical fiber 135. From information associated with the reflected light signals 150, the console 110 may determine (through computation or extrapolation of the wavelength shifts) the physical state of the elongate probe 120.

According to one embodiment of the disclosure, as shown in FIG. 1, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the optical fiber 135 within the elongate probe 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the optical fiber 135 deployed within the elongate probe 120, and (ii) translate the reflected light signals 150 into reflection data (from a data repository 190), namely data in the form of electrical signals representative of the reflected light signals including wavelength shifts caused by strain. The reflected light signals 150 associated with different spectral widths may include reflected light signals 151 provided from sensors positioned in the center core fiber (reference) of the optical fiber 135 and reflected light signals 152 provided from sensors positioned in the periphery core fibers of the optical fiber 135, as described below. Herein, the optical receiver 184 may be implemented as a photodetector, such as a positive-intrinsic-negative “PIN” photodiode, avalanche photodiode, or the like.

Both the light source 182 and the optical receiver 184 are operably connected to the one or more processors 160, which governs their operation. Also, the optical receiver 184 is operably coupled to provide the reflection data (from the data repository 190) to the memory 165 for storage and processing by reflection data classification logic 192. The reflection data classification logic 192 may be configured to: (i) identify which core fibers pertain to which of the received reflection data (from the data repository 190) and (ii) segregate the reflection data stored within the data repository 190 provided from reflected light signals 150 pertaining to similar regions of the elongate probe 120 or spectral widths into analysis groups. The reflection data for each analysis group is made available to state sensing logic 194 for analytics.

According to one embodiment of the disclosure, the state sensing logic 194 is configured to compare wavelength shifts measured by sensors deployed in each periphery core fiber at the same measurement region of the elongate probe 120 (or same spectral width) to the wavelength shift at a center core fiber of the optical fiber 135 positioned along central axis and operating as a neutral axis of bending. From these analytics, the state sensing logic 194 may determine the shape the core fibers have taken in three-dimensional space and may further determine the current physical state of the elongate probe 120 in three-dimensional space for rendering on the display 170.

According to one embodiment of the disclosure, the state sensing logic 194 may generate a rendering of the current physical state of the elongate probe 120, based on heuristics or run-time analytics. For example, the state sensing logic 194 may be configured in accordance with machine-learning techniques to access the data repository 190 with pre-stored data (e.g., images, etc.) pertaining to different regions of the elongate probe 120 in which reflected light from core fibers have previously experienced similar or identical wavelength shifts. From the pre-stored data, the current physical state of the elongate probe 120 may be rendered. Alternatively, as another example, the state sensing logic 194 may be configured to determine, during run-time, changes in the physical state of each region of the optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the optical fiber 135 to the wavelength shift generated by a sensor of the center core fiber at the same cross-sectional region. It is contemplated that other processes and procedures may be performed to utilize the wavelength shifts as measured by sensors along each of the core fibers within the optical fiber 135 to render appropriate changes in the physical state of the elongate probe 120, especially to enable guidance of the elongate probe 120 when positioned multi-core within the patient and at a desired destination within the body.

The console 110 may further include optional electrical signaling logic 181 configured to receive one or more electrical signals from the elongate probe 120. The elongate probe 120 is configured to support both optical connectivity as well as electrical connectivity. The electrical signaling logic 181 receives the electrical signals (e.g., ECG signals) from the elongate probe 120 via the conductive medium. The electrical signal analytic logic 196 may be configured to extract an ECG signal from the electrical signals. The electrical signal analytic logic 196 may further cause an ECG waveform to be portrayed on the display 170.

It is contemplated that other processes and procedures may be performed to utilize the wavelength shifts as measured by sensors along each of the core fibers within the optical fiber 135 to render appropriate changes in the physical state of the probe 120, especially to enable placement and/or guidance of the elongate probe 120 within the patient and at a desired destination within the body. For example, wavelength shifts as measured by sensors along one or more of the core fibers may be based on physical states or conditions of the probe 120 other than or in addition to longitudinal strain experienced by the elongate probe 120. Alternative or additional physical states may include one or more of torsional strain, temperature, motion, fluctuations, oscillations, pressure, or fluid flow adjacent the elongate probe.

Additionally, the console 110 includes a fluctuation logic 195 that is configured to analyze at least a subset of the wavelength shifts measured by sensors deployed in each of the core fibers 137. In particular, the fluctuation logic 195 is configured to analyze wavelength shifts measured by sensors of core fibers 137, where such corresponds to an analysis of the fluctuation of the distal end 122 of the elongate probe 120 or any other section of the elongate probe 120 (or “tip fluctuation analysis”). In some embodiments, the fluctuation logic 195 analyzes the wavelength shifts measured by sensors at a distal end of the core fibers 137. A “tip fluctuation analysis” may include at least a correlation of detected movements of the distal portion 129 of the elongate probe 120 with experiential knowledge comprising previously detected movements (fluctuations), and optionally, other current measurements such as ECG signals. The experiential knowledge may include previously detected movements in various locations within the vasculature (e.g., SVC, Inferior Vena Cava (IVC), right atrium, azygos vein, other blood vessels such as arteries and veins) under normal, healthy conditions and in the presence of defects (e.g., vessel constriction, vasospasm, vessel occlusion, etc.). Thus, the tip fluctuation analysis may result in a confirmation of a location of the distal portion 129 and/or detection of a defect affecting a blood vessel.

It should be noted that the fluctuation logic 195 need not perform the same analyses as the shape sensing logic 194. For instance, the shape sensing logic 194 determines a 3D shape of the elongate probe 120 by comparing wavelength shifts in outer core fibers of a multi-core optical fiber to a center, reference core fiber. The fluctuation logic 195 may instead correlate the wavelength shifts to previously measured wavelength shifts and optionally other current measurements without distinguishing between wavelength shifts of outer core fibers and a center, reference core fiber as the tip fluctuation analysis need not consider direction or shape within a 3D space.

In some embodiments, e.g., those directed at tip location confirmation, the analysis of the fluctuation logic 195 may utilize electrical signals (e.g., ECG signals) measured by the electrical signaling logic 181. For example, the fluctuation logic 195 may compare the movements of a subsection of the elongate probe 120 (e.g., the distal tip) with electrical signals indicating impulses of the heart (e.g., the heartbeat). Such a comparison may reveal whether the distal tip is within the SVC or the right atrium based on how closely the movements correspond to a rhythmic heartbeat.

In various embodiments, a display and/or alert may be generated based on the fluctuation analysis. For instance, the fluctuation logic 195 may generate a graphic illustrating the detected fluctuation compared to previously detected tip fluctuations and/or the anatomical movements of the patient body such as rhythmic pulses of the heart and/or expanding and contracting of the lungs. In one embodiment, such a graphic may include a dynamic visualization of the present medical device moving in accordance with the detected fluctuations adjacent to a secondary medical device moving in accordance with previously detected tip fluctuations. In some embodiments, the location of a subsection of the medical device may be obtained from the shape sensing logic 194 and the dynamic visualization may be location-specific (e.g., such that the previously detected fluctuations illustrate expected fluctuations for the current location of the subsection). In alternative embodiments, the dynamic visualization may illustrate a comparison of the dynamic movements of the subsection to one or more subsections moving in accordance with previously detected fluctuations of one or more defects affecting the blood vessel.

According to one embodiment of the disclosure, the fluctuation logic 195 may determine whether movements of one or more subsections of the elongate probe 120 indicate a location of a particular subsection of the elongate probe 120 or a defect affecting a blood vessel, based on heuristics or run-time analytics. For example, the fluctuation logic 195 may be configured in accordance with machine-learning techniques to access a data store (library) with pre-stored data (e.g., experiential knowledge of previously detected tip fluctuation data, etc.) pertaining to different regions (subsections) of the elongate probe 120. Specifically, such an embodiment may include processing of a machine-learning model trained using the experiential knowledge, where the detected fluctuations serve as input to the trained model and processing of the trained model results in a determination as to how closely the detected fluctuations correlate to one or more locations within the vasculature of the patient and/or one or more defects affecting a blood vessel.

In some embodiments, the fluctuation logic 195 may be configured to determine, during run-time, whether movements of one or more subsections of the elongate probe 120 indicate a location of a particular subsection of the elongate probe 120 or a defect affecting a blood vessel, based on at least (i) resultant wavelength shifts experienced by the core fibers 137 within the one or more subsections, and (ii) the correlation of these wavelength shifts generated by sensors positioned along different core fibers at the same cross-sectional region of the elongate probe 120 to previously detected wavelength shifts generated by corresponding sensors in a core fiber at the same cross-sectional region. It is contemplated that other processes and procedures may be performed to utilize the wavelength shifts as measured by sensors along each of the core fibers 137 to render appropriate movements in the distal portion 129 of the elongate probe 120.

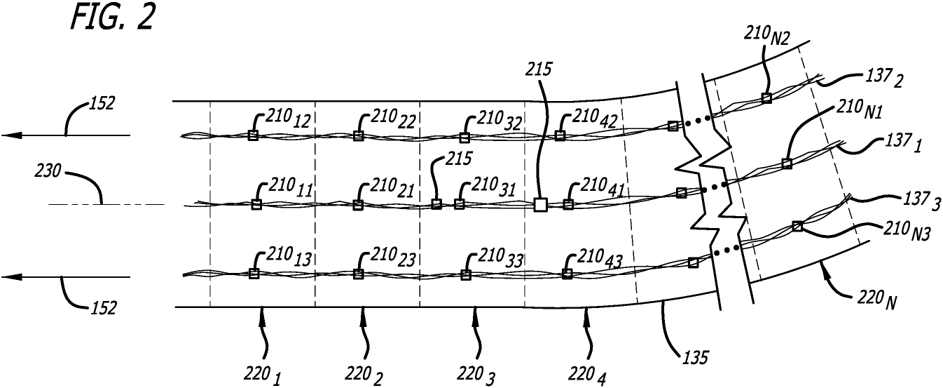

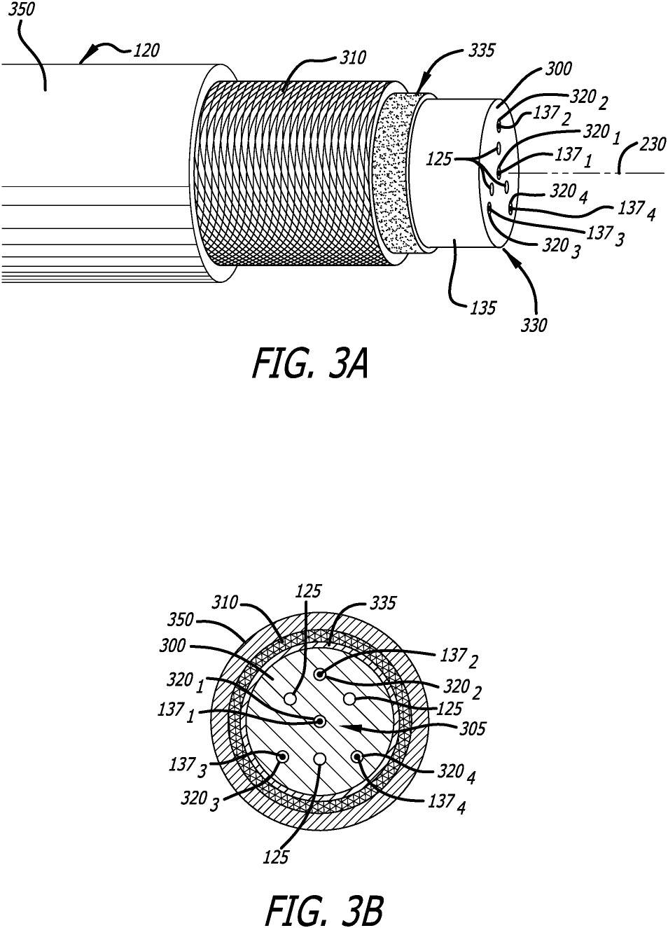

Referring to FIG. 2, an exemplary embodiment of a structure of a section of the elongate probe 120 of FIG. 1 is shown in accordance with some embodiments. The multi-core optical fiber section 200 of the optical fiber 135 depicts certain core fibers 137₁-137_{M} (M>2, M=4 as shown, see FIG. 3A) along with the spatial relationship between sensors (e.g., reflective gratings) 210₁₁-210_{NM} (N2≥2; M≥2) present within the core fibers 137₁-137_{M}, respectively. As noted above, the core fibers 137₁-137_{M} may be collectively referred to as “the core fibers 137.”

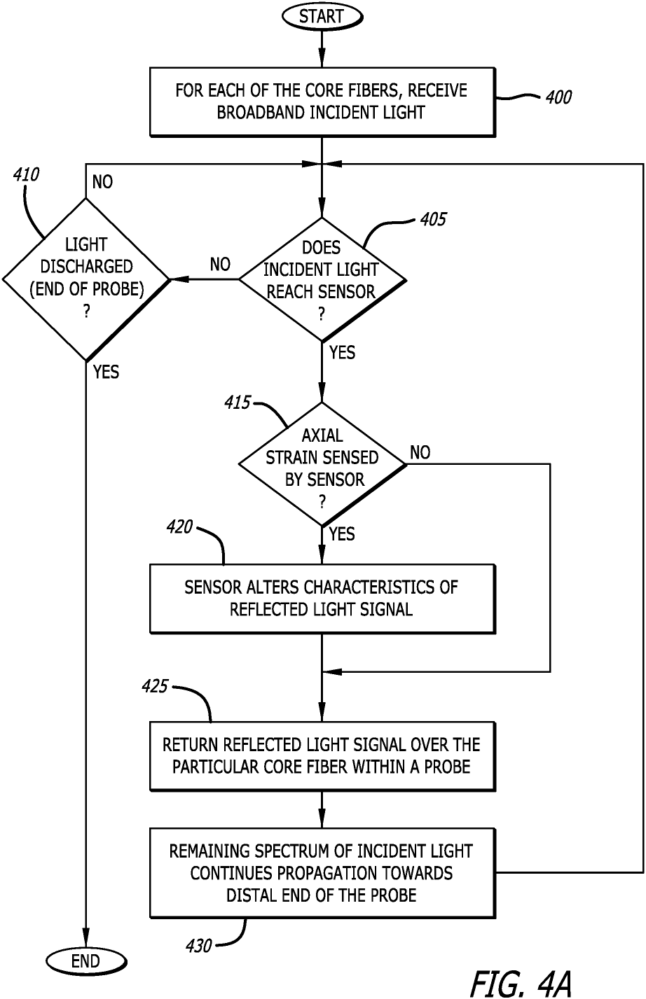

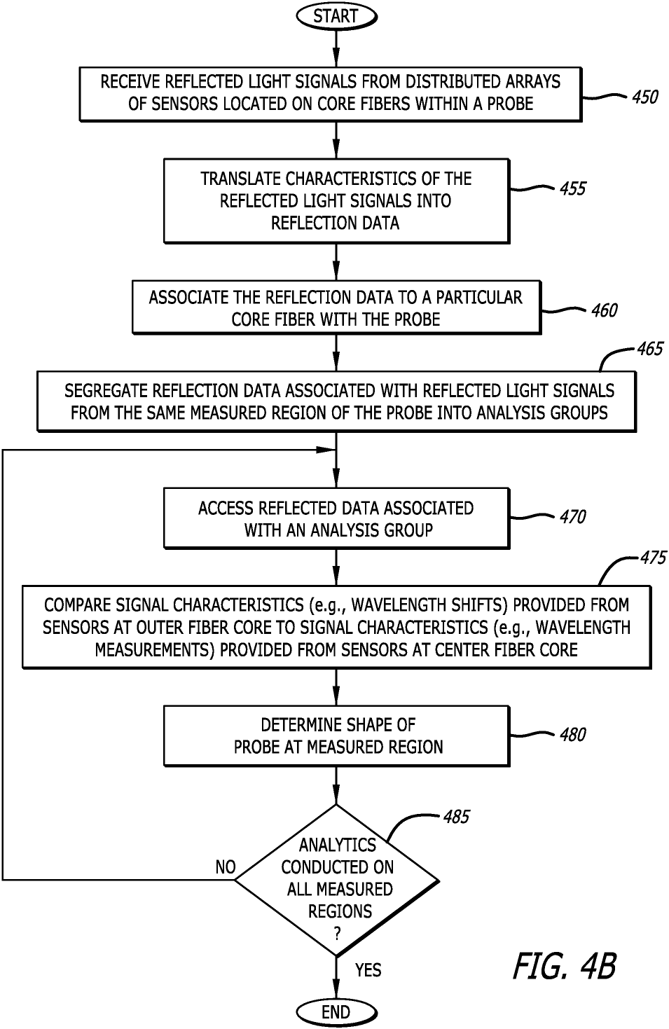

As shown, the section 200 is subdivided into a plurality of cross-sectional regions 220₁-220_{N}, where each cross-sectional region 220₁-220_{N} corresponds to reflective gratings 210₁₁-210₁₄...210_{N1}-210_{N4}. Some or all of the cross-sectional regions 220₁...220_{N} may be static (e.g., prescribed length) or may be dynamic (e.g., vary in size among the regions 220₁... 220_{N}). A first core fiber 137₁ is positioned substantially along a center (neutral) axis 230 while core fiber 137₂ may be oriented within the cladding of the optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the elongate probe 120 (see FIG. 1) is operative, each of the reflective gratings 210₁-210_{N} reflects light for a different spectral width. As shown, each of the gratings 210₁ᵢ 210Ni (1≤i≤M) is associated with a different, specific spectral width, which would be represented by different center frequencies of f ₁... f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the core fibers 137 (and the elongate probe 120) based on wavelength shifts measured from the returned, reflected light. In particular, strain (e.g., compression or tension) applied to the optical fiber 135 (e.g., at least core fibers 137₂-137₃) results in wavelength shifts associated with the returned, reflected light. Based on different locations, the core fibers 137₁-137₄ experience different types and degree of strain based on angular path changes as the elongate probe 120 advances in the patient. Specifically, the core fibers 137₁-137₄ may experience a strain when the curved distal tip 128 becomes less curved.

For example, with respect to the multi-core optical fiber section 200 of FIG. 2, in response to angular (e.g., radial) movement of the elongate probe 120 is in the left-veering direction, the fourth core fiber 137₄ (see FIG. 3A) of the optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the elongate probe 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the optical fiber 135. These degrees of wavelength change may be used to extrapolate the physical state of the elongate probe 120. The reflected light signals 150 are reflected back to the console 110 via individual paths over a particular core fiber 137₁-137_{M}.

In some embodiments, although not required, the optical fiber 135 may include sensors 215, where wavelength shifts as measured by the sensors 215 along the optical fiber 135 may be based on physical states or conditions of the probe 120 that include one or more than a temperature experienced by the elongate probe 120, a pressure exerted on the elongate probe 120, or a fluid flow (e.g., blood flow) adjacent the elongate probe 120. The sensors 215 may located along any of the core fibers 137 or along additional core fibers (not shown). In accordance with the sensors 215, the state sensing logic 194 may be configured to determine one or more of the temperature, the pressure, or the fluid flow.

Referring to FIG. 3A, an exemplary embodiment of the elongate probe 120 of FIG. 1 supporting both an optical and electrical signaling is shown in accordance with some embodiments. Herein, the elongate probe 120 features a centrally located a multi-core optical fiber 135, which includes a cladding 300 and a plurality of core fibers 137₁-137_{M} (M≥2; M=4) residing within a corresponding plurality of lumens 320₁-320_{M}. While the optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the optical fiber 135 and the elongate probe 120 deploying the optical fiber 135.

The optical fiber 135 is encapsulated within a concentric tubing 310 (e.g., braided tubing as shown) positioned over a low coefficient of friction layer 335. The concentric tubing 310, may in some embodiments, feature a “mesh” construction, in which the spacing between the intersecting elements may be selected based on the degree of rigidity/flexibility desired for the elongate probe 120, as a greater spacing may provide a lesser rigidity, and thereby, a more flexible elongate probe 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core3fibers 137₂-137₄, exclusive of the central core fiber 137₁, may be positioned at different areas within a cross-sectional area 305 of the cladding 300 to provide sufficient separation to enable three-dimensional sensing of the optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o′clock), “bottom-left” (8 o′clock) and “bottom-right” (4 o′clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the elongate probe 120, the braided tubing 310 provides mechanical integrity to the multi-core optical fiber 135 The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both the cladding 300 and the braided tubing 310, as shown.

As stated above, the elongate probe 120 includes a number of electrical conductors 125 (e.g., wires) extending along the length of the elongate probe 120. In some embodiments, the electrical conductors 125 may be embedded within the cladding 300 of the optical fiber 135 as shown. In other embodiments, the electrical conductors 125 may be enclosed within the insulating layer 350 in other ways, such as between the friction layer 335 and the braided tubing 310, between the braided tubing 310 and the insulating layer 350 friction, or between the friction layer 335 and the optical fiber 135, for example. In some embodiments, the electrical conductors 125 may include the braided tubing 310. In some embodiments, the electrical conductors 125 may be disposed along an outer surface of the elongate probe 120.

Referring to FIGS. 4A-4B, flowcharts of methods of operations conducted by the medical device system of FIG. 1 to achieve optic three-dimensional shape sensing are shown in accordance with some embodiments. The first micro-lumen is coaxial with the central axis of the probe. The first micro-lumen is configured to retain a center core fiber. Two or more micro-lumen, other than the first micro-lumen, are positioned at different locations circumferentially spaced along the circumferential edge of the probe. For example, two or more of the second plurality of micro-lumens may be positioned at different quadrants along the circumference edge of the probe.

Furthermore, each core fiber includes a plurality of sensors spatially distributed along its length between at least the proximal and distal ends of the probe. This array of sensors is distributed to position sensors at different regions of the core fiber to enable distributed measurements of strain throughout the entire length or a selected portion of the probe. These distributed measurements may be conveyed through reflected light of different spectral widths (e.g., specific wavelength or specific wavelength ranges) that undergoes certain wavelength shifts based on the type and degree of strain, including oscillations of the strain.

According to one embodiment of the disclosure, as shown in FIG. 4A, for each core fiber, broadband incident light is supplied to propagate through a particular core fiber (block 400). Unless discharged, upon the incident light reaching a sensor of a distributed array of sensors measuring strain on a particular core fiber, light of a prescribed spectral width associated with the first sensor is to be reflected back to an optical receiver within a console (blocks 405-410). Herein, the sensor alters characteristics of the reflected light signal to identify the type and degree of strain on the particular core fiber as measured by the first sensor (blocks 415-420). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the probe (blocks 425-430). The remaining spectrum of the incident light may encounter other sensors of the distributed array of sensors, where each of these sensors would operate as set forth in blocks 405-430 until the last sensor of the distributed array of sensors returns the reflected light signal associated with its assigned spectral width and the remaining spectrum is discharged as illumination.

Referring now to FIG. 4B, during operation, multiple reflected light signals are returned to the console from each of the plurality of core fibers residing within the corresponding plurality of micro-lumens formed within a probe. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 450-455). The reflection data classification logic is configured to identify which core fibers pertain to which reflection data and segregate reflection data provided from reflected light signals pertaining to a particular measurement region (or similar spectral width) into analysis groups (block 460-465).

Each analysis group of reflection data is provided to sensing logic for analytics (block 470). Herein, the sensing logic compares wavelength shifts at each outer core fiber with the wavelength shift at the center core fiber positioned along central axis and operating as a neutral axis of bending (block 475). From this analytics, on all analytic groups (e.g., reflected light signals from sensors in all or most of the core fibers), the sensing logic may determine the shape the core fibers have taken in three-dimensional space, from which the sensing logic can determine the current physical state of the probe in three-dimensional space (blocks 480-485).

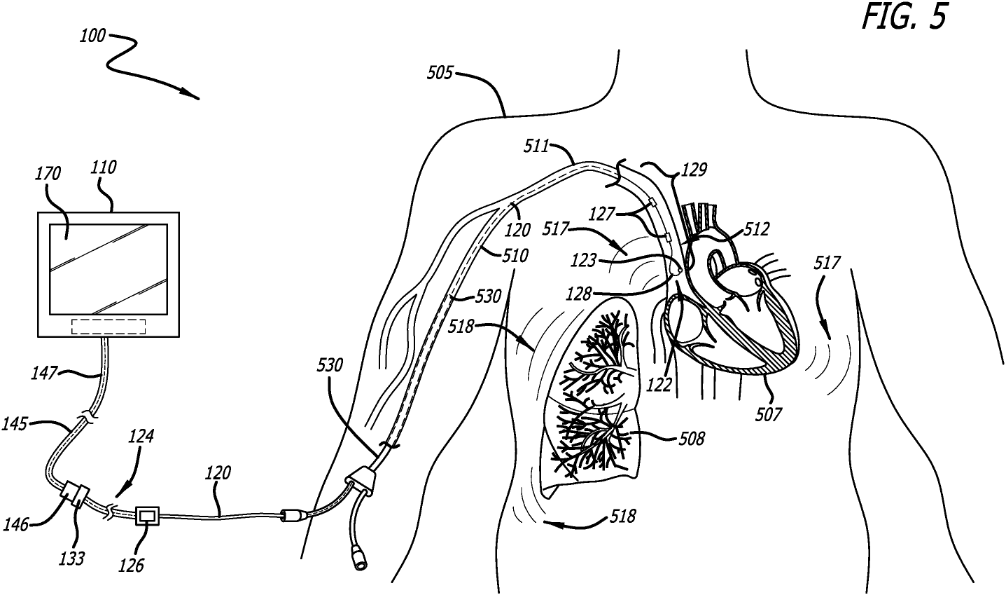

FIG. 5 illustrates an exemplary embodiment of the medical instrument placement system 100 of FIG. 1 during operation and insertion of a catheter into a patient 505. Herein, the elongate probe 120 is advanced to a desired position within the patient vasculature so that a distal end 122 of the elongate probe 120 is proximate the patient’s heart, such as in the lower one-third (⅓) portion of the Superior Vena Cava (“SVC”) for example. During advancement of the elongate probe 120, the elongate probe 120 may pass through different portions of the vasculature. In the illustrated example, the elongate probe 120 passes through the brachial vein 510 and the subclavian vein 511 on its way to the superior vena cava 512. As such, during advancement of the elongate prob 120, the distal portion 129 passed through (i.e., for a period of time resided within) the brachial vein 510 and the subclavian vein 511 on its way to the superior vena cava 512. Following insertion of the elongate probe 120, the catheter 530 may be advanced along the elongate probe 120. In the illustrated example, the catheter 530 is partially advanced along elongate probe 120 on its way toward the superior vena cava 512.

The fluctuation logic 195 of FIG. 1 may be configured to analyze at least a subset of the wavelength shifts measured by sensors deployed in each of the core fibers 137. For example, each core fiber 137 of the elongate probe 120 may be comprised of a plurality of subsections with each subsection including a set of sensors, where the sensors of each subsection may receive an incident light signal and alter the characteristics of the reflected light signal in accordance with detected axial strain. The fluctuation logic 195 may then analyze the wavelength shifts corresponding to the reflected light signal received from a subsection of the elongate probe 120.

Certain organs/tissues within the patient body generate fluctuations (i.e., fluctuating tissue movement), such as the heart 507 and the lungs 508. FIG. 5 shows heartbeat fluctuations 517 generated by the heart 507 and breathing fluctuations 518 generated by the lungs 508. Each of the heartbeat fluctuations 517 and breathing fluctuations 518 may cause portions of the elongate probe 120 to fluctuate when the elongate probe 120 is disposed within the vasculature. In some instances, one portion of the elongate probe 120 may be located within the vasculature so as to fluctuate in accordance with the heartbeat fluctuations 517. Similarly, another portion of the elongate probe 120 may be located within the vasculature so as to fluctuate in accordance with the breathing fluctuations 518.

The fluctuation logic 195 may be configured to determine the location of the elongate probe 120 (or more specifically the location of various portions of the elongate probe 120) within the vasculature. By way of one example, the fluctuation logic 195 may determine the location of the distal portion 129 of the elongate probe 120. As the superior vena cava 512 is adjacent the heart 507, the distal portion 129 may may fluctuate in accordance with the heartbeat fluctuations 517 when the distal portion 129 is disposed within the superior vena cava 512. As such, the fluctuation logic 195 may detect heartbeat fluctuations 517 along the distal portion 129 and thereby determine when the distal portion 129 is disposed within the superior vena cava 512. In some embodiments, the fluctuation logic 195 may notify the user that the distal portion 129 is disposed within the superior vena cava 512.

The electrical signal analytic logic 196 may be configured to determine the position of the tip electrode 123 within the vasculature. More specifically, the electrical signal analytic logic 196 may utilize an ECG signal obtained by the tip electrode 123 to determine a location of the tip electrode 123 within the superior vena cave 512, such as within the lower one-third (⅓) portion of the superior vena cava 512, for example. Thus, ECG signal obtained by the tip electrode 123 serves as an aide in confirming proper placement of elongate probe 120, and thereafter the catheter 530.

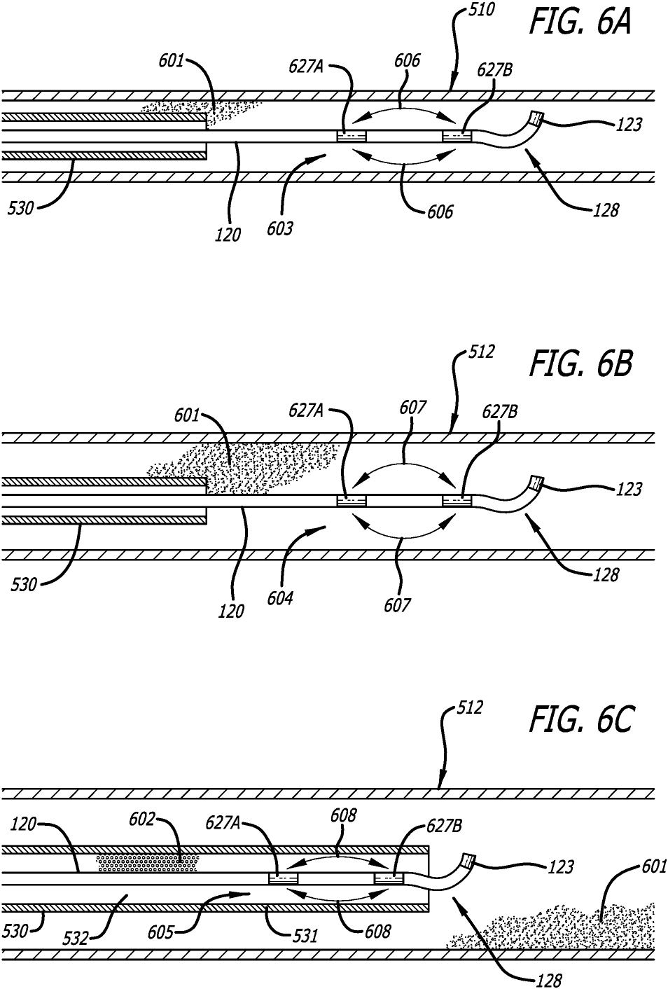

FIGS. 6A-6B illustrate the sensing of impedance between two band electrodes 627A, 627B under various conditions. FIG. 6A illustrates the elongate probe 120 within the brachial vein 510, where the brachial vein 510 has a smaller cross-sectional area than the subclavian vein 511 or the superior vena cave 512. Blood 601 flows along the band electrodes 627A, 627B through an annular flow path 603 defined the brachial vein 510 having the elongate probe 120 disposed therein. A first electrical impedance 606 may generally be defined by (i) the conductivity of the blood 601, (ii) the distance between the band electrodes 627A, 627B, and (iii) a cross-sectional area of the annular flow path 603.

FIG. 6B illustrates the elongate probe 120 within the superior vena cava 512 defining an annular flow path 604 having a greater cross sectional area than the annular flow path 603 of the brachial vein 510. A second electrical impedance 606 may generally be defined by (i) the conductivity of the blood 601, the distance between the band electrodes 627A, 627B, and a cross-sectional area of the annular flow path 603. As the (i) the conductivity of the blood 601 and the distance between the band electrodes 627A, 627B may be constant, the second electrical impedance 607 may be less than the first impedance 606 because the cross-sectional area of the annular flow path 604 is greater than the cross-sectional area of the annular flow path 603.

FIG. 6C illustrates the elongate probe 120 along with the catheter 530 disposed within the superior vena cava 512. The catheter 530 is advanced over the band electrodes 627A, 627B to define an annular flow path 605 between the elongate probe 120 and the catheter wall 531. A third electrical impedance 608 may generally be defined by (i) the conductivity of the fluid 602 with the catheter 530, the distance between the band electrodes 627A, 627B, and a cross-sectional area of the annular flow path 605. As diameter of the catheter lumen 532 is smaller than the diameter of the brachial vein 510 and the diameter of the superior vena cava 512, the cross sectional area of the annular flow path 605 may be less than the cross-sectional areas of the annular flow paths 603, 604. Assuming the fluid 602 has a similar conductivity as the blood 601 and the distance between the band electrodes 627A, 627B is substantially constant, the third electrical impedance 608 may be greater than the first impedance 606 and the second impedance 607 because the cross-sectional area of the annular flow path 605 is less than the cross-sectional areas of the annular flow paths 603, 604.

The electrical signal analytic logic 196 may utilize an impedance signal (an electrical signal related to the impedance between the band electrodes 627A, 627B) to determine a location of the elongate probe 120 within the vasculature. By way of one example, the electrical signal analytic logic 196 may monitor the impedance signal during advancement of the elongate probe 120 along the vasculature. The electrical signal analytic logic 196 may detect a change in the impedance signal when the distal portion 129 (i.e., the band electrodes 627A, 627B) passes from the brachial vein 510 into the subclavian vein 511. In some embodiments, the electrical signal analytic logic 196 may notify the user when the distal portion 129 pass from one vein to another vein. For example, the electrical signal analytic logic 196 may notify the user when the distal portion 129 enters the superior vena cava 512.

The electrical signal analytic logic 196 may utilize an impedance signal to determine a location of the catheter 530 with respect to the elongate probe 120. By way of one example, the electrical signal analytic logic 196 may monitor the impedance signal during advancement of the catheter 530 along the elongate probe 120. The electrical signal analytic logic 196 may detect a change in the impedance signal when the catheter 530 is advanced over the distal portion 129 (i.e., the band electrodes 627A, 627B). In some embodiments, the electrical signal analytic logic 196 may notify the user when the catheter 530 is displaced over (i.e., covers) the distal portion 129. In some instances, the distal portion 129 may be located (i.e., previously positioned) at a desired location for the catheter 530 (or more specifically the distal end of the catheter 530), such as within the lower ⅓^{rd} portion of the superior vena cava 512. As such, by notifying the user when the catheter 530 covers the distal portion 129, the notification may also indicate that the distal end of the catheter 530 is positioned within the lower ⅓^{rd} portion of the superior vena cava 512.

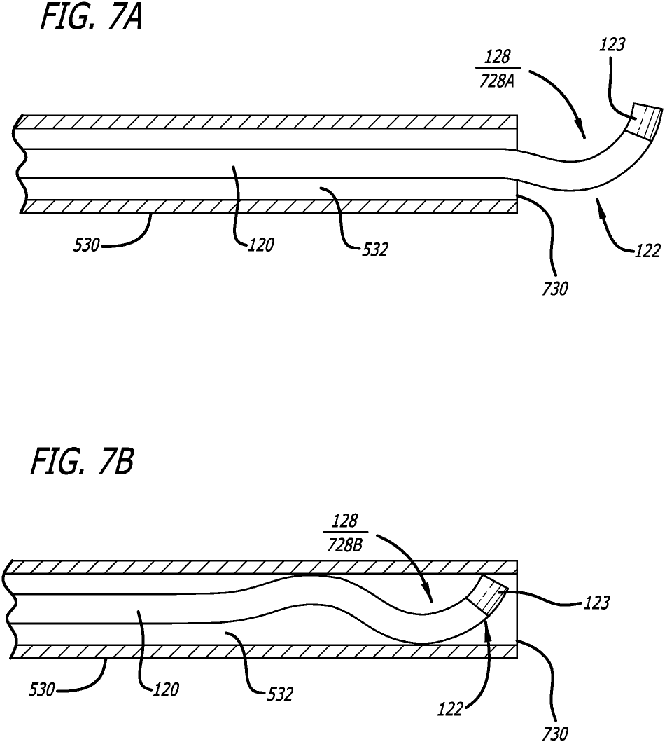

FIGS. 7A-7B illustrate the curved distal tip 128 in two states of bending strain. FIG. 7A illustrates the curved distal tip 128 outside of the catheter lumen 532, where the curved distal tip 128 defines a first bending strain 728A along the curved distal tip 128 consistent with the curved distal tip 128 in the free state.

FIG. 7B illustrates the curved distal tip 128 within of the catheter lumen 532, where the curved distal tip 128 defines a second bending strain 728A along the curved distal tip 128 consistent with the curved distal tip 128 constrained within the catheter lumen 532. As shown, the curved distal tip 128 less curved (i.e., defines a larger radius of curvature) when disposed within the catheter lumen 532 than when the curved distal tip 128 is disposed outside the catheter lumen 532 (FIG. 7A).

The state sensing logic 194 may be configured to detect the change in bending strain between the first bending strain 728A and the second bending strain 728A. As such, the state sensing logic 194 may determine that the catheter 530 covers the curved distal tip 128, i.e., distal end 730 of the catheter 530 is beyond the distal end 122 of the elongate probe 120. Furthermore, the state sensing logic 194 may be configured to notify/alert the user, during advancement of the catheter 530 along the elongate probe 120, when the distal end 730 of the catheter 530 is advanced along the curved distal tip 128. As such, the user may know that the distal end 730 of the catheter 530 is disposed adjacent the distal end of the elongate probe 120. By way of one example, in some instances, the distal portion 129 of the elongate probe 120 may be located (i.e., previously positioned) at a desired location for the catheter 530 (or more specifically the distal end of the catheter 530), such as within the lower ⅓^{rd} portion of the superior vena cava 512. As such, by notifying the user when distal end 730 of the catheter 530 is advanced along the curved distal tip 128, the notification also indicates that the distal end of the catheter 530 is positioned within the lower ⅓^{rd} portion of the superior vena cava 512.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device comprising:
an elongate probe configured for insertion into a patient body, the elongate probe defining a proximal end and a curved distal tip at a distal end;
an optical fiber extending along the elongate probe from the proximal end to the distal end, the optical fiber including one or more core fibers extending along a longitudinal length of the optical fiber, each of the one or more core fibers including a plurality of sensors distributed along the longitudinal length and each sensor of the plurality of sensors being configured to (i) reflect a light signal of a different spectral width based on received incident light at proximal end, and (ii) change a characteristic of the reflected light signal based on a condition experienced by the optical fiber along the curved distal tip.

2. The device of claim 1, wherein:
the elongate probe is configured for advancement along a vasculature of the patient body such that the elongate probe experiences fluctuations due to fluctuating movement of patient body tissue, and
the fluctuations define the condition experienced by the optical fiber along the curved distal tip.

3. The device of claim 1, wherein:
the elongate probe is configured for insertion within a lumen of a vascular catheter;
the curved distal tip includes a flexibility in bending such that, upon disposition of the curved distal tip within the lumen, a radius of curvature of the curved distal tip is increased defining a bending strain along the curved distal tip; and
the bending strain defines the condition experienced by the optical fiber along the curved distal tip.

4. The device of claim 1, wherein the elongate probe includes a guidewire.

5. The device of claim 1, wherein the elongate probe includes a number of electrical conductors extending along the elongate probe from the proximal end to the distal end.

6. The device of claim 5, wherein:
the elongate probe includes a tip electrode at the distal end,
the tip electrode is coupled with at least one of the number of electrical conductors, and
the tip electrode is configured to obtain an ECG signal from the patient body.

7. The device of claim 5, wherein:
the elongate probe includes a number of band electrodes disposed along the elongate probe,
each band electrode is coupled with at least one of the number of electrical conductors, and
the band electrodes are configured to obtain an electrical impedance between two or more band electrodes.

8. A medical system comprising:
a medical device comprising:
an elongate probe configured for insertion into a patient body, the elongate probe defining a proximal end and a curved distal tip at a distal end;
an optical fiber extending along the elongate probe from the proximal end to the distal end, the optical fiber including one or more core fibers extending along a longitudinal length of the optical fiber, each of the one or more core fibers including a plurality of sensors distributed along the longitudinal length and each sensor of the plurality of sensors being configured to (i) reflect a light signal of a different spectral width based on received incident light at proximal end, and (ii) change a characteristic of the reflected light signal based on condition experienced by the optical fiber; and
a console operatively coupled with the medical device at the proximal end, the console including one or more processors, and a non-transitory computer-readable medium having stored thereon logic that, when executed by the one or more processors, causes operations of the system that include determining a physical state of the curved distal tip, wherein determining the physical state includes:
providing an incident light signal to the optical fiber;
receiving reflected light signals of different spectral widths of the incident light by one or more of the plurality of sensors along the curved distal tip; and
processing the reflected light signals associated with the one or more core fibers to determine the physical state of the curved distal tip.

9. The system of claim 8, wherein the physical state includes fluctuations along the curved distal tip, the fluctuations caused by fluctuating tissue movement within the patient body.

10. The system of claim 9, wherein the fluctuating tissue movement is caused by a heartbeat.

11. The system of claim 8, wherein the physical state includes a bending strain along the curved distal tip.

12. The system of claim 11, wherein the bending strain along the curved distal tip is caused by disposition of the curved distal tip within a lumen of a vascular catheter.

13. The system of claim 8, wherein:
the elongate probe includes a number of electrical conductors extending along the elongate probe from the proximal end to the distal end; and
the operations of the system include receiving an electrical signal from one or more of the electrical conductors.

14. The system of claim 13, wherein:
the elongate probe includes a tip electrode at the distal end,
the tip electrode is coupled with at least one of the number of electrical conductors, and
the electrical signal includes an ECG signal.

15. The system of claim 13, wherein:
the elongate probe includes a number of band electrodes disposed along the elongate probe,
each band electrode is coupled with at least one of the number of electrical conductors, and
the electrical signal includes an impedance between two or more of the number of band electrodes.

16. A method of placing a catheter within a vasculature of a patient body, comprising:
providing a guidewire including:
an optical fiber extending along the guidewire, the optical fiber operatively coupled with a console at the proximal end of the guidewire; and
a number of electrical conductors extending along the guidewire, the electrical conductors operatively coupled with the console at the proximal end;
advancing the guidewire along a vascular pathway of the patient body;
determining a position of the guidewire within the vascular pathway based on one or more of a first optical signal received by the console from the guidewire or a first electrical signal received by the console from the guidewire;
advancing the catheter along the guidewire, the guidewire disposed within a lumen of the catheter; and
determining a position of the catheter with respect to the guidewire based on one or more of a second optical signal or a second electrical signal.

17. The method of claim 16, wherein:
the optical fiber includes one or more core fibers extending along a longitudinal length of the optical fiber, each of the one or more core fibers including a plurality of sensors distributed along the longitudinal length and each sensor of the plurality of sensors being configured to (i) reflect a light signal of a different spectral width based on received incident light at proximal end, and (ii) change a characteristic of the reflected light signal based on condition experienced by the optical fiber;
the first optical signal includes reflected light signals of different spectral widths from one or more of the plurality of sensors based on received incident light at the proximal end;
the different spectral widths are defined by a fluctuation of the optical fiber; and
the fluctuation is caused by fluctuating tissue movement adjacent the vascular pathway.

18. The method of claim 16, wherein:
the guidewire includes a curved distal tip,
the second optical signal includes reflected light signals of different spectral widths from one or more of the plurality of sensors disposed along the curved distal tip based on received incident light at the proximal end,
the different spectral widths are defined by a change in a bending strain along the curved distal tip, and
the change in bending strain is caused by advancing the catheter along the curved distal tip.

19. The method of claim 16, wherein:
the guidewire includes a tip electrode at the distal end,
the tip electrode is coupled with at least one of the number of electrical conductors, and
the first electrical signal includes an ECG signal obtained by the tip electrode.

20. The method of claim 16, wherein:
the guidewire includes a plurality of band electrodes disposed along the guidewire;
each band electrode is coupled with at least one of the number of electrical conductors;
the first electrical signal is defined by a change in electrical impedance between two or more band electrodes;
the change in electrical impedance is defined by a change in an annular fluid pathway extending along the two or more band electrodes; and
the change in the annual fluid pathway is caused by advancing the guidewire between two portions of the vascular pathway, the two portions having different cross-sectional areas.

21. The method of claim 16, wherein:
the guidewire includes a plurality of band electrodes disposed along the guidewire,
each band electrode is coupled with at least one of the number of electrical conductors,
the second electrical signal is defined by a change in electrical impedance between two or more band electrodes,
the change in electrical impedance is defined by a change in an annular fluid pathway extending along the two or more band electrodes, and
the change in the annual fluid pathway is caused by advancing the catheter over the two or more band electrodes.
